(19) United States
(12) Patent Application Publication
(10) Pub. No.: US 2026/0315266 A1
(43) Pub. Date: Oct. 15, 2026

(54) METHOD FOR ACCELEROMETER INPUT FOR OSTOMY LEAKAGE DETECTION SYSTEM

(71) Applicant: Hollister Incorporated, Libertyville, IL (US)

(72) Inventors: Kyle A. Matthews, Chapel Hill, NC (US); Anthony B. Smith, Durham, NC (US); Scott E. Liddle, Raleigh, NC (US)

(73) Assignee: Hollister Incorporated, Libertyville, IL (US)

(21) Appl. No.: 19/473,984

(22) PCT Filed: May 14, 2024
(86) PCT No.: PCT/US2024/029270

§ 371 Date: Oct. 9, 2025

Related U.S. Application Data
(60) Provisional application No. 63/510,676, filed on Jun. 28, 2023.

(51) Int. Cl. A61F 5/44; A61F 5/445

(52) U.S. Cl. CPC A61F 5/4404; A61F 5/445

(57) ABSTRACT

A method for accelerometer input for an ostomy leakage detection system. The method may be applied to a wearable device that can obtain a leakage data, determine a leakage level based on the leakage data, obtain accelerometer data, detect a double tap input based on the accelerometer data, and silence a haptic feedback leak level alert output based on the double tap input. The leakage data may include a leakage detected on at least one ring of a sensing accessory. The leakage level can indicate information about the leakage detected. The haptic feedback leak level alert discreetly notifies a user about the leakage detected.

### BACKGROUND

The present disclosure pertains to a method for accelerometer input for an ostomy leakage detection system.

An ostomy pouch system typically includes a pouch formed from opposing sidewalls defining an internal collection area, an inlet opening for receiving a stoma, and an ostomy appliance for attaching the pouch to a user. The ostomy appliance may include, for example, an ostomy barrier of a one-piece pouch system, which is attached to one of the pouch sidewalls proximate an inlet opening, or a faceplate for a two-piece pouch system configured to releasably engage a pouch, and a barrier ring. The ostomy appliance may include a skin barrier material for adhering to and sealing against user's peristomal skin surrounding the stoma.

The ostomy appliance may be susceptible to ostomy effluent leakage, and the seal formed between the skin barrier material and the user may weaken. Oftentimes, the user may be unaware of, or cannot easily assess, an extent of weakening in the seal. Thus, the user may not become aware of a weakened seal, and consequently, the ostomy effluent may cause skin health complications and may leak through to an exterior of the ostomy appliance.

Leakage detection systems may include an electronic device that processes leak data, detects leakage in ostomy appliances, and notifies a user of detected leakage. However, the electronic device may be attached to a user, and it may be difficult for a user to discreetly input user input. For example, a user may want to check the leakage status of the ostomy appliance without accessing the electronic device.

Accordingly, it is desirable to provide a method for discreetly inputting user input to an electronic device for an ostomy leakage detection system.

### BRIEF SUMMARY

A method for accelerometer input for an ostomy leakage detection system is provided according to various embodiments.

In a first aspect of the present disclosure, a method for accelerometer input for an ostomy leakage detection system is provided.

In an embodiment, the method may be applied to a wearable device. The wearable device may obtain a leakage data. The leakage data may include a leakage detected on at least one ring of a sensing accessory. The wearable device may also determine a leakage level based on the leakage data. The leakage level indicates information about the leakage detected. The wearable device may obtain accelerometer data. The wearable device may detect a double tap input based on the accelerometer data. The wearable device may silence a haptic feedback leak level alert output based on the double tap input. The haptic feedback leak level alert may discreetly notify a user about the leakage detected.

In an embodiment, the wearable device may also obtain a system context mode. The system context mode may include a leakage detected mode.

In an embodiment, the wearable device may also obtain a system context mode. The system context mode may include a training mode. The wearable device may further silence a training mode haptic feedback leak level alert output based on the double tap input. In such an embodiment, the wearable device may also output a double tap notification to a mobile device.

In an embodiment, the wearable device may also obtain a system context mode. The system context mode may include a leak not detected. The wearable device may further obtain a battery level alert based on the double tap input. The battery level alert may include a haptic feedback alert.

In an embodiment, the wearable device may also obtain a system context mode. The system context mode may include a sensor not detected mode. The wearable device may further output a power state alert based on the double tap input. The power state alert may include a light alert.

In an embodiment, the wearable device may also obtain an activity mode. The wearable device may further adjust a data sample rate of the leakage data based on the activity mode. In such an embodiment, the activity mode may include a high activity mode. In a similar embodiment, the activity mode may include a swimming activity mode.

In an embodiment, the wearable device may also store the accelerometer data in a first in first out (FIFO) buffer. The wearable device may further obtain the accelerometer data from the FIFO buffer. In such an embodiment, the FIFO buffer may include 32 samples.

In an embodiment, the wearable device may also fetch the accelerometer data from the FIFO buffer. The wearable device may further clear the FIFO buffer.

In a second aspect of the present disclosure, a computing device is provided. The computing device may include one or more processors, a non-transitory computer-readable memory storing instructions executable by the one or more processors. The one or more processors may be configured to obtain a leakage data. The leakage data may include a leakage detected on at least one ring of a sensing accessory. The one or more processors may also be configured to determine a leakage level based on the leakage data. The leakage level indicates information about the leakage detected. The one or more processors may further be configured to obtain accelerometer data. The one or more processors may be configured to detect a double tap input based on the accelerometer data. The one or more processors may also be configured to silence a haptic feedback leak level alert output based on the double tap input. The haptic feedback leak level alert may discreetly notify a user about the leakage detected.

In an embodiment, one or more processors may also be configured to obtain a system context mode. The system context mode may include a leakage detected mode.

In an embodiment, one or more processors may also be configured to obtain a system context mode. The system context mode may include a training mode. The one or more processors may also be configured to silence a training mode haptic feedback leak level alert output based on the double tap input.

In an embodiment, one or more processors may also be configured to output a double tap notification to a mobile device.

In an embodiment, one or more processors may also be configured to store the accelerometer data in a FIFO buffer. The one or more processors may also be configured to obtain the accelerometer data from the FIFO buffer. In such an embodiment, the FIFO buffer may include 32 samples.

In an embodiment, one or more processors may also be configured to fetch the accelerometer data from the FIFO buffer. The one or more processors may also be configured to clear the FIFO buffer.

In a third aspect of the present disclosure, a non-transitory computer-readable storage medium having stored therein instructions is provided. When the instructions are executed by one or more processors of the apparatus, the instructions may cause the apparatus to The foregoing general description and the following detailed description are examples only and are not restrictive of the present disclosure.

### BRIEF DESCRIPTION OF THE DRAWINGS

The benefits and advantages of the present embodiments will become more readily apparent to those of ordinary skill in the relevant art after reviewing the following detailed description and accompanying drawings, wherein:

FIG. 1A is an illustration of an ostomy system according to an embodiment.

FIG. 1B is a front view of an ostomy system attached to a user, according to an embodiment.

FIG. 2A is a body-side perspective view of a sensor circuit, according to an embodiment.

FIG. 2B is a partially enlarged body-side perspective view of a sensor circuit, according to an embodiment.

FIG. 3 is a top-side perspective view of a wearable device in a closed position according to an embodiment.

FIG. 4 is a top cross-section perspective view of the wearable device of FIG. 2 in an open position.

FIG. 5 is a flow diagram illustrating a method for accelerometer input for an ostomy leakage detection system, according to an embodiment.

FIG. 6 is a flow diagram illustrating a method for accelerometer input for an ostomy leakage detection system, according to another embodiment.

FIG. 7 is a schematic illustration of a computing environment, according to an embodiment.

### DETAILED DESCRIPTION

While the present disclosure is susceptible of embodiment in various forms, there is shown in the drawings and will hereinafter be described presently preferred embodiments with the understanding that the present disclosure is to be considered an exemplification and is not intended to limit the disclosure to the specific embodiments illustrated. The words “a” or “an” are to be taken to include both the singular and the plural. Conversely, any reference to plural items shall, where appropriate, include the singular. The words “first,” “second,” “third,” and the like may be used in the present disclosure to describe various information, such information should not be limited to these words. These words are only used to distinguish one category of information from another. The directional words “top,” “bottom,” up,“ ”down,“ front,” “back,” and the like are used for purposes of illustration and as such, are not limiting. Depending on the context, the word “if” as used herein may be interpreted as “when” or “upon” or “in response to determining.”

The present disclosure provides a method for accelerometer input for ostomy leakage detection systems. An accelerometer can consist of one or more sensors that measure the mechanical forces acting on it, such as gravity or motion. These sensors can convert the mechanical forces into electrical signals for tracking the movement and/or orientation of the device or triggering an event based on the detected acceleration. Ostomy leakage detection systems can be configured to detect ostomy effluent leakage under a skin barrier and to alert a user. Ostomy leakage detection systems can provide multiple benefits to the user. For example, the system can allow the user to intervene and change a skin barrier and/or ostomy pouch system before a leak progresses which can cause embarrassment and inconvenience to the user. Further, ostomy leakage detection systems can assist in maintaining a user's skin health by detecting a leakage in its early stage to prevent prolonged skin exposure to ostomy effluent, which can lead to skin health complications. Ostomy leakage detection systems can also support a user's emotional well-being by reducing anxiety associated with a risk of leakage. Ostomy leakage detection systems may be applied to an ostomy barrier of a one-piece pouch system or a faceplate for a two-piece pouch system.

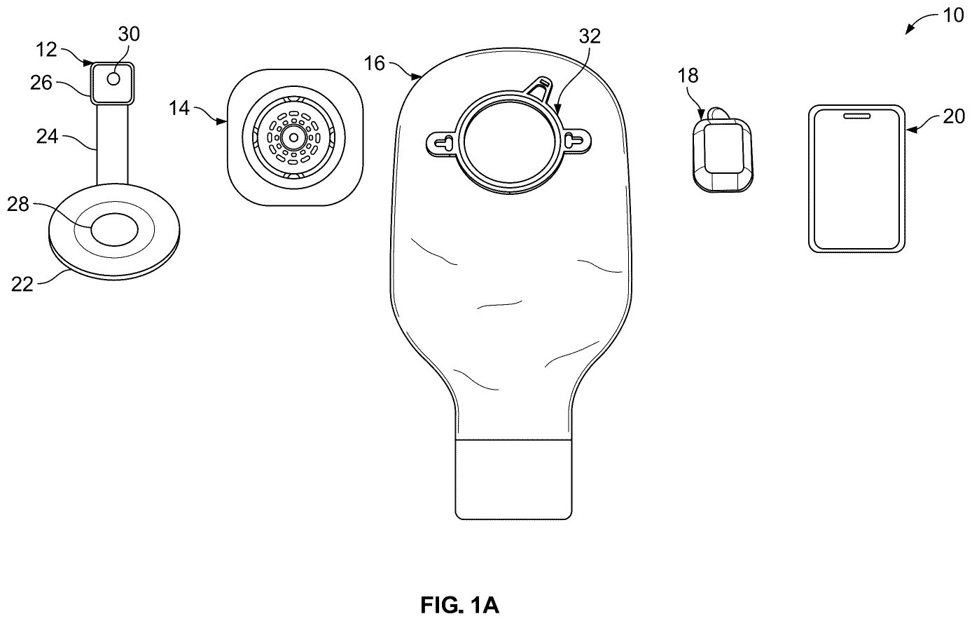

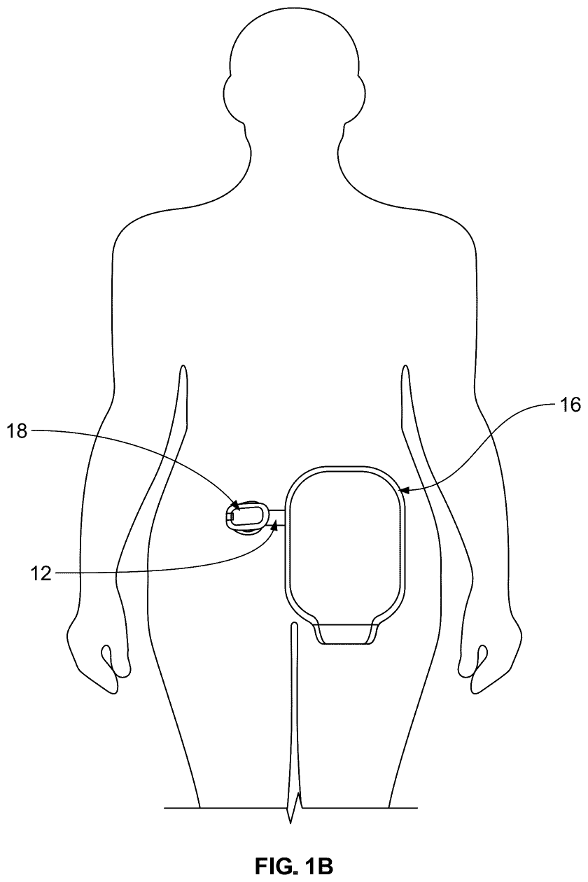

FIG. 1A illustrates an ostomy two-piece pouch system 10. According to the example embodiment shown schematically in FIG. 1A, the ostomy system 10 can generally include a sensing accessory 12 with a tail end 13, an ostomy barrier appliance 14, an ostomy pouch 16 with an ostomy barrier coupling member 18, a wearable device 20, and a mobile electronic device 21.

FIG. 1B illustrates the ostomy pouch system 10 attached to a user according to the example embodiment. According to example embodiments shown schematically in FIG. 1B, an end of the sensing accessory 12 can be attached to the user and can surround a stoma. The tail end 13 can be attached to the wearable device 20. The sensing accessory 12 can be designed to detect leakage or moisture around the stoma. The ostomy barrier appliance 14 can be attached over the sensing accessory 12. The ostomy pouch 16 can be connected to the ostomy barrier appliance 14 using the ostomy barrier coupling member 18. The wearable device 20 can be attached to the user with, for example, an adhesive patch.

According to example embodiments, the ostomy leakage detection system may comprise three subsystems-the sensing accessory 12, the wearable device 20, and a mobile application on the mobile electronic device 21. The sensing accessory 12 may be provided as an accessory for an ostomy pouch system. The sensing accessory 12 can include sensors for detecting the presence of ostomy effluent. The sensing accessory 12 may be configured to communicate leakage detection signals to the wearable device 20. For example, the wearable device 20 can generate leakage data based on the leakage detection signals from the sensing accessory 12. The leakage data can include whether leakage is detected and in what ring and quadrant the leakage is detected.

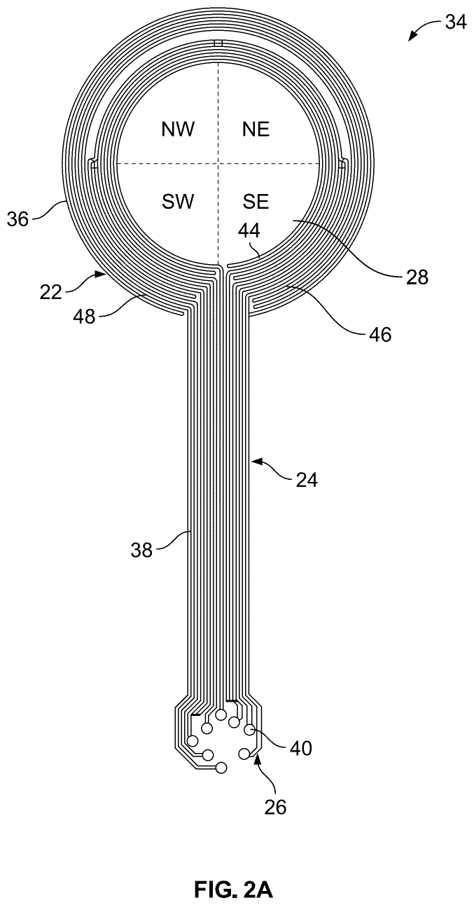

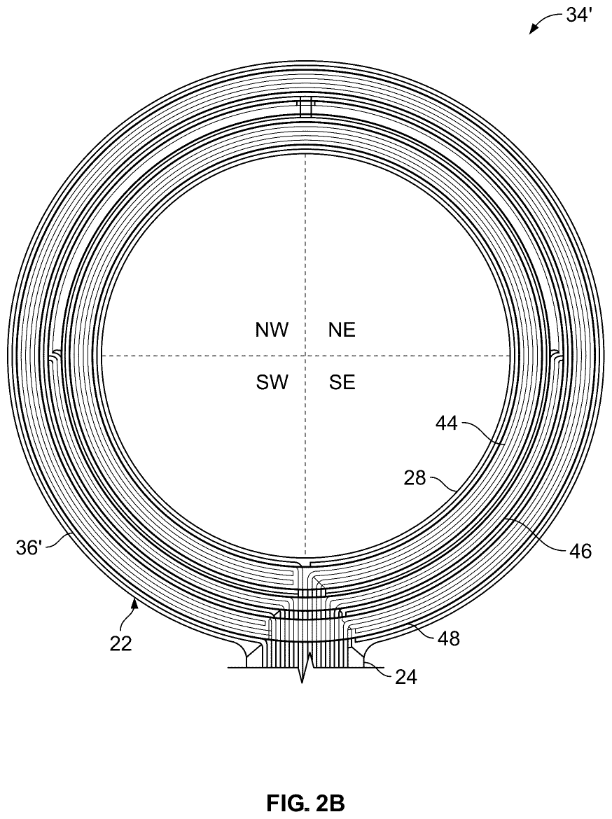

FIG. 2A shows a body-side view of a sensor circuit 34. According to the example embodiment shown schematically in FIG. 2A, the sensor circuit 34 can generally include a conductive sensor 36, a conductive trace 38, and a connection point 40. The sensor circuit 34 can be located on the sensing region 22, the tail region 24, and the connection region 26. The conductive sensor 36 can be configured to detect a leak around a stoma. The conductive trace 38 can be located on the tail region 24 and may be configured to connect the conductive sensor 36 to the connection point 40. The connection point 40 can be located on the connection region 26 and may be configured to connect to a connection pad on the wearable device 20. The sensing region 22 can be broken into four quadrants NE, SE, NW, SW.

According to example embodiments, the conductive sensor 36 can include multiple pairs of conductive traces that can be arranged in radial sensing levels (or rings) with levels broken into the four quadrants NE, SE, NW, SW of sensor pairs for detecting the direction of fluid and moisture progression. The radial sensing levels can include a first radial sensing level (or first ring 44) located near the inlet opening 28, a second radial sensing level (or second ring 48) located near the outer edge of the sensing region 22, and one or more radial sensing levels (or rings 46) located between the first and second radial sensing levels. The conductive traces can be adjacent to ground traces so that resistance may be measured between the two for detecting fluid and moisture. In another embodiment, the conductive traces can detect fluid and moisture based on resistance, resonance frequency or the like.

The conductive sensor 36 may be arranged in a predetermined pattern in the sensing region 22. For example, the sensor circuit 34 may be generally arranged in a circular or semi-circular pattern. Other suitable patterns are envisioned as well, such as an oval or oblong pattern, or other closed or substantially closed loop pattern. The sensor circuit 34 in the sensing region 22 may be arranged at one or more radial distances from the inlet opening 28. For example, the sensor circuit 34 may include a plurality of electrically conductive traces arranged at a plurality of different radial distances from the inlet opening 28. The sensor circuit 34 may include conductive traces and conductive pads or points that may be formed by printing on a circuit substrate using a conductive ink via a conventional printing process, for example, screen printing.

FIG. 2B shows a body-side view of a sensor circuit 34′. According to the example embodiment shown schematically in FIG. 2B, the sensor circuit 34′ can include a conductive sensor 36′. The conductive sensor 36′ can include a first radial sensing level 44 (or first ring) located near the inlet opening 28, a second radial sensing level 46 (or second ring) located between the first radial sensing level and a third radial sensing level, and the third radial sensing levels 48 (or third ring) located near the outer edge of the sensing region 22.

### Leakage Detection

In an embodiment, the wearable device 20 can detect a leakage. For example, the leakage data can be generated by the wearable device 20 using electrical signals from the sensing accessory 12. The leakage data can include whether a leakage is detected and in what ring and quadrant the leakage is detected. The leakage level can then be determined based on predefined ring thresholds. The predefined ring thresholds can help indicate to the user how a leak is progressing from the inlet opening 28. The leakage level can be determined on the wearable device 20, the mobile application on the mobile electronic device 21, or other computing system that has access to the leakage data.

In an embodiment, the predefined ring thresholds can include a threshold for each ring detecting a leakage. For example, a first ring threshold can be met when a first ring detects a leakage. The leakage level can then be determined to be a level 1. In another example, a second ring threshold can be met when two rings detect a leakage, and the leakage level can be determined to be a level 2. In yet another example, a third ring threshold can be met when all rings detect a leakage, and the leakage level can be determined to be a level 3.

In another embodiment, the predefined ring thresholds can be set not only based on the number of rings detecting a leakage, but which rings detect such a leakage. For example, a leakage level 1 can include a predefined ring threshold for an inner ring that indicates that a leak is detected only on the inner ring of the sensing region 22, a leakage level 2 can include a predefined ring threshold for an inner ring and middle ring that indicates that the leak is detected on the inner ring and is progressing onto the middle ring, and the leakage level 3 can include a predefined ring threshold for an inner ring, middle ring, and outer ring that indicates that the leak is detected on the inner ring and has progressed onto the middle ring and the outer ring.

In another embodiment, a moisture level can be determined. The leakage data can include whether moisture is detected and in what ring and quadrant the moisture is detected. A moisture level can then be determined based on predefined ring thresholds. For example, the moisture level can include a predefined ring threshold for an outer ring that indicates that moisture is detected only on the outer ring of the sensing region 22.

### Wearable Device

The wearable device 20 may be configured to perform at least some processing of the leakage detection signals and alert a user of a leakage event. The wearable device 20 may be configured to electronically communicate with the mobile application through a wired or wireless communication system. Such electronic communications may include raw data acquired from the sensing accessory 12 or summarized leak data generated from processing the raw data. The wearable device 20 may also communicate system conditions, such as the presence of a sensing accessory, a faulty sensor, or a battery state. The mobile application may be a digital subsystem and/or software application installed and able run on the mobile electronic device 21. The mobile application may be configured to further process leak detection data and provide an alert or other information about an ostomy appliance to a user.

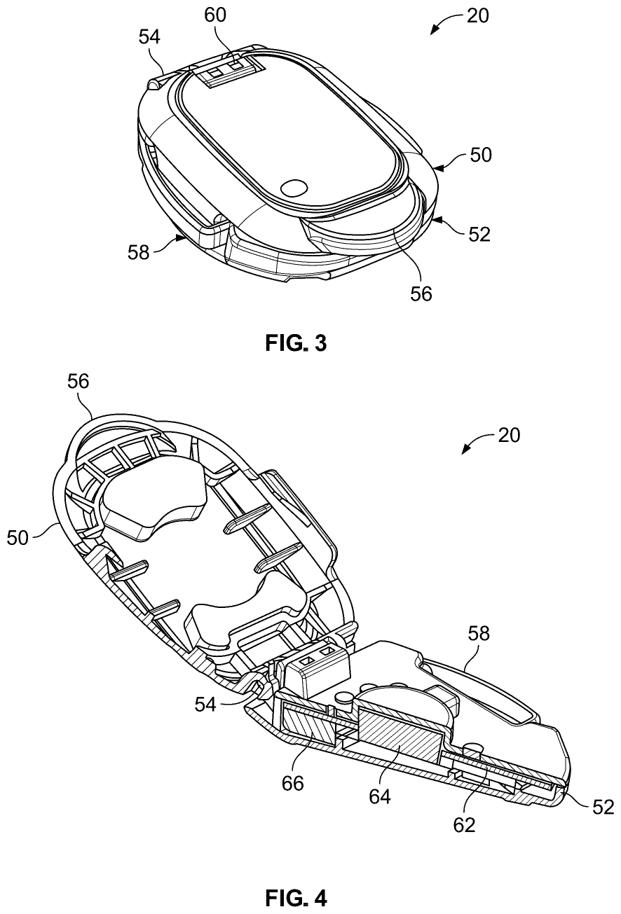

Referring back to the figures, FIGS. 3 and 4 show wearable device 20 according to an embodiment. The wearable device 20 may generally include a top housing 50, a bottom housing 52, a hinge 54, a lid member 56, a latching mechanism 58, and one or more light indicators 60.

FIG. 3 shows a wearable device 20 in a closed position. The lid member 56 can be located on an edge of the top housing 50 and opposite the hinge 54. According to the example embodiment shown schematically in FIG. 3, the lid member 56 can aid in opening and closing the wearable device 20. The lid member 56 may be a lid lift touch point. The latching mechanism 58 can secure the wearable device 20 in the closed position. The one or more light indicators 60 can be viewable from the top housing 50 and configured to indicate conditions of wearable device 20 such as a system status, battery status, and error messages. In another embodiment, the one or more light indicators 60 can be positioned at the sides of the wearable device 20 or other areas where they can be visible to a user while wearing the wearable device 20. For example, one or more light indicators 60 can light the entire top housing 50 by having translucent material as the cover and positioning the one or more light indicators 60 to light the cover.

FIG. 4 shows a top perspective cross-section view of the wearable device 20 in an open position. According to the example embodiment shown schematically in FIG. 4, the wearable device 20 can include a printed circuit board (PCB) 62, a battery 64, and a haptic motor 66 within a sealed electronic enclosure. The haptic motor 66 can provide a haptic buzzer for silent or discreet notifications where the user, while wearing the wearable device 20 will feel vibrations from the haptic motor 66 in the form of pulses. The battery 64 or haptic motor 66 may be aligned within a center raised member for space savings. The PCB 62 can include a processing unit, a communication unit, and electronic connectors that connect with the one or more sensor contact pads 40. The PCB 62 can analyze signals received from the sensing accessory 12, communicate with a mobile device 21 or a charging dock, and alert a user through sound and vibration, as well as one or more lights to indicate a system status. The wearable device 20 can also include an accelerometer that can be on the PCB 62 or electrically coupled to it. The position of the accelerometer can be where it can best detect movement, orientation, and taps by the user. For example, the accelerometer may be positioned at a center area of the top housing 50. In an embodiment, for linear tap acceleration input detection, the accelerometer can be positioned near to the center of the tap force application location. Additionally, the accelerometer may be positioned near the axis of impact to allow for a rotational component to be detected. However, the accelerometer can be placed anywhere on the PCB or housing.

### Accelerometer Input

In one or more embodiments, the accelerometer can be used to measure a triaxial acceleration data of the wearable device 20. The triaxial acceleration data can be generated by sensors in the accelerometer. Sensors can include, for example, piezoelectric sensors, capacitive sensors, piezoresistive sensors, and MEMS (Micro-Electro-Mechanical Systems) sensors that can be used in accelerometers for measuring motion, orientation, and vibrations. When the accelerometer sensors measure triaxial acceleration data that meet a wakeup threshold, the accelerometer can store the measured triaxial acceleration data in a buffer storage system in the form of samples. The wearable device 20 can then fetch and analyze the samples. For example, the accelerometer sensors can generate triaxial acceleration data at 100 Hz and store the resulting samples on an on-board circular or ring buffer storage system. In an embodiment, the buffer storage system can be a First In First Out (FIFO) buffer. The FIFO buffer can hold up to 32 samples. When the FIFO buffer fills up (which can happen almost immediately), the oldest sample is dropped to make room for the newest sample.

In an embodiment, the wearable device 20 can fetch samples from the FIFO buffer to determine an orientation of the wearable device 20 relative to gravity, to receive a user's input in the form of taps, and to determine a current activity of a user. In an embodiment, the accelerometer can clear the FIFO buffer once the wearable device 20 fetches the samples. Clearing the samples on the FIFO buffer can allow for low power detection. For example, clearing the FIFO buffer after a fetch, allows for low power implementation of a tap detection, the power is sufficiently low that the tap detection can run natively on the wearable device itself. In another embodiment, the wearable device may have a tap detection algorithm running on the accelerometer itself. For example, a manufacturer provided tap detection algorithm. The wearable device may just receive a detection output from the accelerometer without needing to read the FIFO (e.g. the accelerometer detects the taps on its own, and merely signals to the wearable device that a tap has been detected).

In an embodiment, the wearable device 20 can fetch samples from the FIFO buffer after a wakeup interrupt is received form the accelerometer. The wakeup interrupt can be generated by the accelerometer sensors when a most recent sample goes above a wakeup threshold. The wakeup threshold can be set to a value that includes a tap detected or an orientation change. The wakeup interrupt allows the wearable device 20 not to have to constantly poll or fetch samples from the FIFO buffer, which would otherwise require a large amount of power. This can be significant, as the accelerometer can be configured to generate new samples at 100 Hz. At that sample rate, the battery life of the wearable device 20 can be very poor. With the wakeup interrupt, the wearable device 20 only needs to fetch samples after the wakeup interrupt is triggered.

In an embodiment, the wearable device 20 can determine a user input in the form of taps on the wearable device 20. Taps can include, for example, a double tap, where the user taps the top housing 50 twice, one right after the other. Other tap schemes can include a triple tap and a single-double tap that includes a single tap followed by a double tap. In an embodiment, the double tap is detected within the 32 samples in the FIFO buffer. In another embodiment, the double tap can be determined with a FIFO buffer less than 32 samples to confirm that the double tap occurred within a threshold time. The threshold time can ensure that the user is trying to input a double tap. In another embodiment, the double tap can be determined after fetching a first set of samples that includes a first detected tap, fetching a second or more set of samples that includes a second detected tap, and determining the time between the two taps. In an embodiment, it can be determined that the time between the two taps falls within a threshold time window to assure a double tap is detected. The threshold time window can be set so that a user can input a double tap without feeling like the wearable device is unresponsive or slow. The threshold time window can be between, for example, 100 ms to 400 ms from the first tap and second tap.

In an embodiment, when the wearable device 20 detects a double tap, a double tap flag is set. The double tap flag allows the wearable device 20 to take into account the user input during operation. For example, the wearable device 20 can take into account the double tap when outputting haptic feedback in order to silence or stop the haptic feedback output.

The wearable device 20 can use the double tap input to execute certain functions. In an embodiment, a system status function may be enabled by a double-tap input. The system status function can include outputting an alert to convey information to the user about the system status. The alert can include haptic feedback by the haptic motor 66, light notifications on the one or more light indicators 60, sound notifications on a speaker or other visual, audio or haptic notifications such as on the mobile device 21.

In an embodiment, the system status function can include a request for a moisture state of the sensing accessory 12. The wearable device 20 can respond to the request for the moisture state of the sensing accessory 12 by outputting a haptic feedback leak level alert based on a determined leakage level. For example, the haptic feedback can include a haptic vibration at a set interval and pattern according to the leak severity (one haptic pulse per leak level, with a lull time between pulse trains). In another embodiment, the system status function can include a request for a battery level. The wearable device 20 can respond to the request for the battery level by outputting a haptic feedback battery level alert based on a determined level of the battery 64. For example, the wearable device 20 can determine a battery level equal to 50% and output a normal haptic feedback battery level alert that includes a soft haptic pulse. In another example, the wearable device 20 can determine a battery level equal to 5% and output a critical haptic feedback battery level alert that includes three strong and short haptic pulses to convey to the user the battery requires immediate charging.

In another embodiment, a silence function may be enabled by the double-tap input. The double tap input may also allow the user to silence an unprompted alert by the wearable device 20, indicating a change in system status. For example, the progression from no moisture to Level 1 moisture. This input may be relayed to other parts of the system, such as a software application hosted on the mobile device 21.

In another embodiment, the wearable device 20 can respond to a double-tap input based on a system context mode. The system context mode can be a mode in which the wearable device 20 is operating. For example, the system context mode can be a training mode where a leakage can be simulated to train the user on how to use the wearable device 20 with the ostomy leakage detection system.

In the training mode, the user can be trained on how to understand alerts output by the wearable device 20 and how to enter user input into the wearable device 20. For example, the training mode trains the user on how the haptic feedback output feels for a given severity of a leakage, and how to interact with the device via taps. This is instructive, as many users are not used to tapping on a device, nor are they used to only receiving feedback via haptics from a device. Both of these use-flows (input and output) are required for the wearable device 20, as they help provide a discreet means of interaction. The training is also instructive in that a specific double-tap cadence is needed to help reduce the likelihood of a missed-double-tap. The tap cadence can be trained to help assist the user obtain a acceptable tap frequency, where the time between the first and second tap is not too slow or not too fast. In the training mode, the wearable device 20 can provide a haptic vibration at a set interval to mimic a leak event of a specified severity. The training mode can be entered upon receipt of a BLUETOOTH Low Energy (BLE) command from the mobile application on the mobile electronic device 21. When a training mode is active and a double tap is detected by the wearable device 20, the training haptic pattern (training mode haptic feedback leak level alert) will be shut off or silenced and the mobile electronic device 21 will be notified (via double tap notification) that the user double tapped the wearable device 20.

In another embodiment, the system context mode can include a leakage detected mode. The wearable device 20 can provide a haptic vibration or pulse at a set interval and pattern according to the leak severity (one haptic pulse per leak level, with a lull time between pulse trains). Once a user double taps the wearable device 20, these leak alerts are silenced. The alert can be silenced on both the mobile electronic device 21 and the wearable device 20, as the silence level can also be transmitted to the mobile electronic device 21.

In another embodiment, the system context mode can include a no leakage detected mode. The no leakage detected mode includes a connected sensor. In an embodiment, the wearable device 20 can respond to a double tap by providing a normal haptic alert output. The normal haptic alert output can convey to the user that the ostomy leakage detection system is working properly. In another embodiment, the wearable device can respond to the double tap by providing a battery level as discussed above. In another embodiment, the wearable device can respond to the double tap by providing a connection level that conveys the level of connection between the wearable device and the mobile electronic device 21. The no leakage detected mode can include other alerts based on user settings on the mobile electronic device 21.

In another embodiment, the system context mode can include a sensor not connected mode. The wearable device 20 can respond to a double tap input with a light notification on the one or more light indicators 60 or a speaker on the wearable device 20. The light notification (power state alert) can represent a power state of the wearable device 20. The power state can be output using a color that corresponds to battery low or battery critical level. For example, a green color can be output for a normal battery level, a yellow for a low battery level, and a red for a critical battery level. A separate light notification can represent the sensor state. The sensor state can include a connected state and a not connected state. For example, a blue color can be output for a disconnected sensor state.

In an embodiment, the accelerometer may also be used to collect data on the ostomy leakage detection system, which may further be used to adjust how system data is processed or communicated. For example, the orientation of the wearable may reflect the orientation of a moisture detection sensor within the system. In this case, the static acceleration field of gravity would indicate the orientation of the wearable. Dynamic accelerations may be indicative of user activity, such as running, walking, swimming or sleeping. This information could be factored into the assessment of system conditions or system alerts.

In another embodiment, the wearable device 20 can respond to an activity mode based on the accelerometer data. The activity mode can include a high activity mode. If the sensing accessory 12 is connected and the activity is high, such as in running or swimming, the wearable device 20 could adjust the leak detection algorithm ‘on the fly’ to account for a greater likelihood of leaking. For example, the wearable device 20 can adjust the data sample rate. For example, the data sample rate may be set lower to account for a mild activity mode and increased when a high activity mode is detected. In an embodiment, the sample rate could be reduced during known sleep times, or a low-active level. For example, if the device accelerometer does not sense much movement (acceleration) over a certain period of time, it can modify the behavior of the wearable device sample rate to save energy/power by setting the sample rate to a low level. Additionally, the wearable device 20 could also adjust to detect more moisture present due to sweat instead of a leakage, and avoid a fake leakage detection.

In another embodiment, the activity mode can be a mild activity mode. If the sensing accessory 12 is connected and activity is mild, such as when the user is walking, the wearable device 20 can adjust the data sample rate. For example, the data sample rate may be set to account for a high activity mode and lowered when a mild activity mode is detected. In an example, the wearable device can also use the orientation of the accelerometer (in concert with a detection movement) to determine if the user is sleeping and modify the sample rate to save even more power. In another example, if it is detected that the user is in a high-active level, for example, running, the wearable device can change into a ‘high risk of leak’ mode where the sample rate can be set to a high rate where leakage detection is prioritized over energy use. In another example, if it is detected that the user is in mid-active level, for example, walking, the sample rate or leak detection algorithm parameters can be set to a normal or mid-level that balances energy use with leakage detection.

In another embodiment, the activity mode can be a no activity mode. If the sensing accessory 12 is connected and the wearable device 20 detects no movement, such as when the user is sitting down or just standing and there is no tap input, the wearable device 20 can use the no activity detected to update a night mode. The night mode can be a mode where the ostomy leakage detection system is operating at night when the user is sleeping. The wearable device 20 can use the no activity detected to adjust the night mode settings (start and end time) without the user having to specify when they go to bed or wake up.

In another embodiment, the activity mode can be a swimming mode. The swimming mode can include the sensing accessory 12 connected, and the wearable device 20 detecting a user is swimming. The swimming mode can turn off leak alerting and set the wearable device 20 to prepare for water ingress recovery. Water ingress recovery can include the wearable device 20 requiring time to dry. It can also prompt the user to remove the wearable device 20 when swimming through haptic alerts, light notifications, and sound alerts.

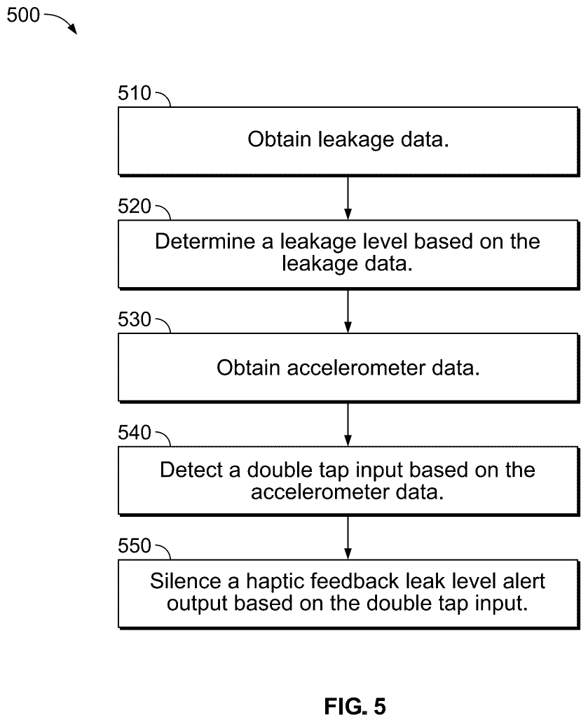

FIG. 5 shows an example of a method 500 for accelerometer input for an ostomy leakage detection system. The method may be applied to a computing device such as a wearable device, mobile device, personal computer or server.

In step 510, the wearable device can obtain leakage data. The leakage data can include a leakage detected on at least one ring of a sensing accessory.

In step 520, the wearable device can determine a leakage level based on the leakage data. The leakage level can inform a user about the severity of the leakage detected. For example, the leakage level can be a leakage level 1 that may not be severe but should be noted by the user. In another example, the leakage level can be a leakage level 2, that may signal a progressing leakage that should be inspected by the user. In yet another example, the leakage level can be a leakage level 3, that may signal a severe leakage that requires changing the ostomy pouch system before a leak progresses and causes inconvenience to the user.

In step 530, the wearable device can obtain accelerometer data.

In step 540, the wearable device can detect a double tap input based on the accelerometer data.

In step 550, the wearable device can silence a haptic feedback leak level alert output based on the double tap input. The haptic feedback leak level alert discreetly notifies a user about the leakage detected. In an embodiment, the wearable device 20 can silence an unprompted alert indicating a change in system status, such as the progression from no moisture to Level 1 moisture. This input may be relayed to other parts of the system such as a software application hosted on the mobile device 21.

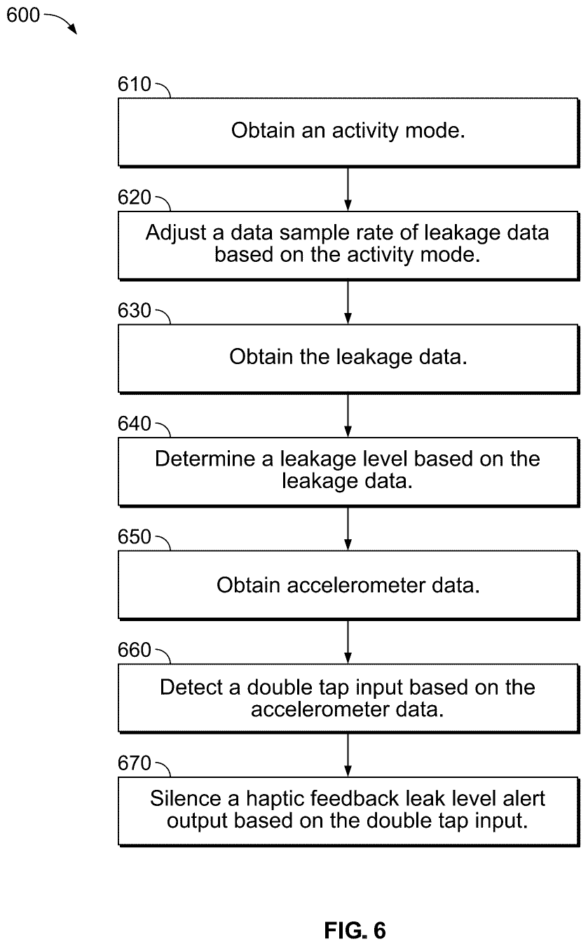

FIG. 6 shows an example of a method 600 for accelerometer input for an ostomy leakage detection system. The method may be applied to a computing device such as a wearable device, mobile device, personal computer or server.

In step 610, the wearable device can obtain an activity mode. The activity mode can be determined by analyzing the accelerometer data to determine the movement and orientation of the system. For example, a high activity mode can include an accelerometer detecting large movement. In another example, a swimming activity mode can include a high or normal activity mode and a slanted orientation indicating the system is in a lying down position (90 degrees from upright orientation).

In step 620, the wearable device can adjust a data sample rate of a leakage data based on the activity mode. The wearable device can adjust a data sample rate by communicating with the accelerometer to change the data sample rate at which it reads generated data. For example, the accelerometer may change the data sample rate from 100 Hz to 50 Hz to lower the data sample rate.

In step 630, the wearable device can obtain the leakage data. The leakage data can include a leakage detected on at least one ring of a sensing accessory.

In step 640, the wearable device can determine a leakage level based on the leakage data. The leakage level can inform a user about the severity of the leakage detected. For example, the leakage level can be a leakage level 1 that may not be severe but should be noted by the user. In another example, the leakage level can be a leakage level 2, that may signal a progressing leakage that should be inspected by the user. In yet another example, the leakage level can be a leakage level 3, that may signal a severe leakage that requires the changing of the ostomy pouch system before a leak progresses and causes embarrassment and inconvenience to the user.

In step 650, the wearable device can obtain accelerometer data. The wearable device can obtain accelerometer data from the FIFO buffer.

In step 660, the wearable device can detect a double tap input based on the accelerometer data. The wearable device can detect a double tap input based on the accelerometer data having samples that indicate two taps recorded within a threshold time. The threshold time can range, for example, between 0.20-1 second. In another embodiment, the threshold time can be set using a bluetooth data link, thus allowing the user to configure the threshold themselves. In another embodiment, the threshold time can be set using a user's demographics to determine an appropriate threshold time. For example, if the user is older, the wearable device can set the threshold time higher (for example, 1 second) to account for slower reaction time by the user.

In other embodiments, the threshold time can be set or configured based from the mobile app by a user input, the mobile app using information about the user (i.e. demographics or past use), from the wearable device through user input, or from the wearable device using past data collected by the accelerometer (i.e. the device detects a number of slow double taps that failed and updates the threshold time).

In step 670, the wearable device can silence a haptic feedback leak level alert output based on the double tap input. The haptic feedback leak level alert can discreetly notify a user about the leakage detected. The wearable device can silence a haptic feedback leak level alert by stopping the haptic motor 66.

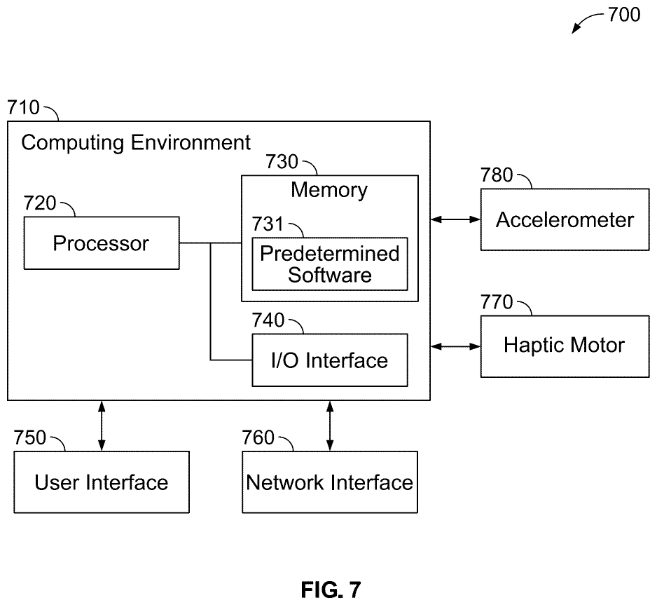

FIG. 7 shows an example of a computing system 700 that can be part of the wearable device 20. According to example embodiments shown schematically in FIG. 7, the computing system 700 can include a computing environment 710, a user interface 750, a network interface 760, a haptic motor 770, and an accelerometer 780. The computing environment 710 can include a processor 720, a memory 730, and an I/O interface 740. The computing environment 710 can be coupled to the user interface 750, network interface 760, haptic motor 770, and accelerometer 780 through the I/O interface 740. The PCB 62 can include the computing environment 710.

The processor 720 can typically control the overall operations of the computing environment 710, such as the operations associated with data acquisition, data processing, and data communications. The processor 720 can include one or more processors to execute instructions to perform all or some of the steps in the above-described methods. Moreover, the processor 720 can include one or more modules that facilitate the interaction between the processor 720 and other components. The processor may be or include a Central Processing Unit (CPU), a microprocessor, a single chip machine, a graphical processing unit (GPU) or the like.

The memory 730 can store various types of data to support the operation of the computing environment 710. Memory 730 can include predetermined software 731. Examples of such data comprise instructions for any applications or methods operated on the computing environment 710, raw data, leak data, moisture data, resistance values, etc. The memory 730 may be implemented by using any type of volatile or non-volatile memory devices, or a combination thereof, such as a static random-access memory (SRAM), an electrically erasable programmable read-only memory (EEPROM), an erasable programmable read-only memory (EPROM), a programmable read-only memory (PROM), a read-only memory (ROM), a magnetic memory, a flash memory, a magnetic or optical disk.

The I/O interface 740 can provide an interface between the processor 720 and peripheral interface modules, such as a RF circuitry, external port, proximity sensor, audio and speaker circuitry, video and camera circuitry, microphone, accelerometer, display controller, optical sensor controller, intensity sensor controller, other input controllers, keyboard, a click wheel, buttons, and the like. The buttons may include but are not limited to, a home button, a power button, and volume buttons.

The user interface 750 can include a speaker, lights, display or other similar technologies for communicating with the user.

Network interface 760 provides communication between the processing unit, an external device, mobile device, and a webserver (or cloud). The communication can be done through, for example, WIFI or BLUETOOTH hardware and protocols. The network interface 760 can be within the computing environment or connected to it.

The haptic motor 770 can include a haptic feedback controller or driver that controls the haptic motor 66 and can cause a vibration or pulse on the wearable device 20. The vibration or pulse on the wearable device 20 can be physically felt by the user when worn or can generate a noise when the wearable device 20 is not worn but on a surface (for example, a table). The haptic feedback controller or driver can send electrical signals to control the haptic motor 770 and cause vibrating pulses.

The accelerometer 780 can include an electromechanical device or other similar technologies that measures a physical acceleration experienced by an object on one, two, or three axes. The accelerometer 780 can include, for example, capacitive plates attached to springs that move internally as acceleration forces act upon the sensor.

In some embodiments, there is also provided a non-transitory computer-readable storage medium comprising a plurality of programs, such as comprised in the memory 730, executable by the processor 720 in the computing environment 710, for performing the above-described methods. For example, the non-transitory computer-readable storage medium may be a ROM, a RAM, or the like.

The non-transitory computer-readable storage medium has stored therein a plurality of programs for execution by a computing device having one or more processors, where the plurality of programs when executed by the one or more processors, cause the computing device to perform the above-described method for motion prediction.

In some embodiments, the computing environment 710 may be implemented with one or more application-specific integrated circuits (ASICs), digital signal processors (DSPs), digital signal processing devices (DSPDs), programmable logic devices (PLDs), field-programmable gate arrays (FPGAs), graphical processing units (GPUs), controllers, micro-controllers, microprocessors, or other electronic components, for performing the above methods.

What is claimed is:

1. A method for accelerometer input for an ostomy leakage detection system comprising:
obtaining a leakage data, wherein the leakage data comprises a leakage detected on at least one ring of a sensing accessory;
determining a leakage level based on the leakage data, wherein the leakage level indicates information about the leakage detected;
obtaining accelerometer data;
detecting a double tap input based on the accelerometer data; and
silencing a haptic feedback leak level alert output based on the double tap input, wherein the haptic feedback leak level alert output discreetly notifies a user about the leakage detected.

2. The method of claim 1, further comprising:
obtaining a system context mode, wherein the system context mode comprises a leakage detected mode.

3. The method of claim 1, further comprising:
obtaining a system context mode, wherein the system context mode comprises a training mode; and
silencing a training mode haptic feedback leak level alert output based on the double tap input.

4. The method of claim 3, further comprising:
outputting a double tap notification to a mobile device.

5. The method of claim 1, further comprising:
obtaining a system context mode, wherein the system context mode comprises a leak not detected; and
outputting a battery level alert based on the double tap input, wherein the battery level alert comprises a haptic feedback alert.

6. The method of claim 1, further comprising:
obtaining a system context mode, wherein the system context mode comprises a sensor not detected mode; and
outputting a power state alert based on the double tap input, wherein the power state alert comprises a light alert.

7. The method of claim 1, further comprising:
obtaining an activity mode; and
adjusting a data sample rate of the leakage data based on the activity mode.

8. The method of claim 7, wherein the activity mode comprises a high activity mode.

9. The method of claim 7, wherein the activity mode comprises a swimming activity mode.

10. The method of claim 1, wherein obtaining accelerometer data comprises:
storing the accelerometer data in a first in first out (FIFO) buffer; and
obtaining the accelerometer data from the FIFO buffer.

11. The method of claim 10, wherein the FIFO buffer comprises 32 samples.

12. The method of claim 10, wherein obtaining the accelerometer data from the FIFO buffer comprises:
fetching the accelerometer data from the FIFO buffer; and
clearing the FIFO buffer.

13. A computing device, comprising:
one or more processors;
a non-transitory computer-readable storage medium storing instructions executable by the one or more processors, wherein the one or more processors are configured to:
obtain a leakage data, wherein the leakage data comprises a leakage detected on at least one ring of a sensing accessory;
determine a leakage level based on the leakage data, wherein the leakage level indicates information about the leakage detected;
obtain accelerometer data;
detect a double tap input based on the accelerometer data; and
silence a haptic feedback leak level alert output based on the double tap input, wherein the haptic feedback leak level alert output discreetly notifies a user about the leakage detected.

14. The computing device of claim 13, wherein the one or more processors are further configured to:
obtain a system context mode, wherein the system context mode comprises a leakage detected mode.

15. The computing device of claim 13, wherein the one or more processors are further configured to:
obtain a system context mode, wherein the system context mode comprises a training mode; and
silence a training mode haptic feedback leak level alert output based on the double tap input.

16. The computing device of claim 15, wherein the one or more processors are further configured to:
output a double tap notification to a mobile device.

17. The computing device of claim 13, 16, wherein the one or more processors configured to obtain accelerometer data are further configured to:
store the accelerometer data in a first in first out (FIFO) buffer; and
obtain the accelerometer data from the FIFO buffer.

18. The computing device of claim 17, wherein the FIFO buffer comprises samples.

19. The computing device of claim 17, wherein the one or more processors configured to obtain the accelerometer data from the FIFO buffer are further configured to:
fetch the accelerometer data from the FIFO buffer; and
clear the FIFO buffer.

20. A non-transitory computer-readable storage medium storing a plurality of programs for execution by a computing device having one or more processors, wherein the plurality of programs, when executed by the one or more processors, cause the computing device to perform acts comprising:
obtaining an activity mode;
adjusting a data sample rate of a leakage data based on the activity mode;
obtaining the leakage data, wherein the leakage data comprises a leakage detected on at least one ring of a sensing accessory;
determining a leakage level based on the leakage data, wherein the leakage level indicates information about the leakage detected;
obtaining accelerometer data;
detecting a double tap input based on the accelerometer data; and
silencing a haptic feedback leak level alert output based on the double tap input, wherein the haptic feedback leak level alert output discreetly notifies a user about the leakage detected.
